(19) United States
(12) Patent Application Publication
(10) Pub. No.: US 2026/0263768 A1
(43) Pub. Date: Sep. 10, 2026

(54) Skincare Therapy Device

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Lu Zhijian, Shenzhen (CN); Meng Xiaoting, Shenzhen (CN); Zhu Yongqi, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/558,765

(22) Filed: Mar. 6, 2026

(30) Foreign Application Priority Data
Mar. 7, 2025 (CN) 2025102736446
Mar. 7, 2025 (CN) 2025204063889
Aug. 14, 2025 (CN) 2025217315799
Nov. 24, 2025 (CN) 2025117367675
Nov. 24, 2025 (CN) 2025225004857
Jan. 27, 2026 (CN) 2026101145496
Feb. 2, 2026 (CN) 2026201522149
Feb. 5, 2026 (CN) 2026201766158

(51) Int. Cl. A61M 37/00; A45D 34/04

(52) U.S. Cl. CPC A61M 37/0015; A45D 34/041; A45D 2200/205; A61M 2037/0061

(57) ABSTRACT

A handheld physiotherapy device integrates a rotatable sleeve with a replaceable microneedle assembly, internal phototherapy lamp plates, and a thermoelectric cooling and heating assembly. A liquid-dispensing system, comprising a container bottle, switch component, liquid outlet channel, with a spherical dispensing ball and elastic bias, and an electronic control valve, provides metered delivery of cosmetic or medicinal liquids to smearing portions and microneedles. Motion detection photoelectric or alternative sensors synchronize dispensing, phototherapy, and optional vibration to operate only during active rolling. The device includes battery power, charging interface, circuit board with firmware safety interlocks, removable limiting covers and interchangeable rollers for microneedle, massage or smearing-only functions, and thermal and mechanical designs that permit combined hot-and-cold therapy, accurate dosing, easy maintenance, and reduced leakage and contamination.

### TECHNICAL FIELD

The present invention relates to a handheld physiotherapy device, and more particularly, to a roller skincare device having a rotatable sleeve component with motion-responsive flow control.

### BACKGROUND OF THE INVENTION

Handheld skincare devices that combine rolling applicators with liquid dispensing are known in the art. Such devices generally include a roller head that is rolled across the skin surface and a reservoir or container for storing a cosmetic or medicinal liquid. In use, the liquid is delivered from the reservoir to the roller surface and applied to the skin as the roller is moved across the treatment area.

Some known devices incorporate microneedle assemblies on the roller surface to create microchannels in the stratum corneum, thereby enhancing the absorption of topically applied formulations into the dermis. Other known devices incorporate light-emitting elements, such as LEDs, in or adjacent to the roller to provide phototherapy to the skin during rolling. Still other devices employ airless pump mechanisms or squeeze-based dispensing to deliver liquid from a container to the roller surface.

However, conventional roller skincare devices suffer from several drawbacks. In many existing designs, the liquid outlet remains open regardless of whether the roller is in use. As a result, when the device is not being used or when the container is inverted during handling or storage, the liquid can flow out of the outlet under the action of gravity, leading to waste of the skincare product and contamination of the device. Devices that rely on manual pumping or squeezing to dispense liquid require the user to perform separate dispensing and rolling actions, which can be inconvenient and can result in inconsistent dosing.

Furthermore, existing devices that combine microneedle therapy with liquid application typically require the user to apply the liquid to the skin first manually and then separately perform the microneedle treatment, or vice versa. This multi-step process is cumbersome and reduces treatment efficiency. Similarly, devices that provide phototherapy or cooling functions generally do so independently of the liquid dispensing and rolling operations, requiring the user to manage multiple functions separately.

There is accordingly a need for a physiotherapy device that can automatically control the flow of liquid in response to the rolling motion of the applicator, thereby preventing unintended leakage when the device is not in use while ensuring consistent liquid delivery during active treatment. There is also a need for a device that integrates multiple therapeutic modalities, such as microneedle therapy, phototherapy, and cooling, with the liquid dispensing and rolling functions in a single compact handheld instrument.

### OBJECTS OF THE INVENTION

Some objects of the invention are as follows:

An object of the present invention is to provide a physiotherapy device that automatically controls the flow of liquid from a container bottle to a rolling applicator in response to detected rotation of the applicator, thereby preventing unintended leakage when the device is not in use.

Another object of the present invention is to provide a physiotherapy device having a motion detection element, such as a photoelectric sensor, that detects rotation of a sleeve component relative to a mounting base and outputs a detection signal to a circuit board for controlling an electronic control valve.

Another object of the present invention is to provide a physiotherapy device having a ball assembled in an outlet tank and sealed with a groove wall thereof, wherein rotation of a rolling applicator assembly drives the ball to rotate and form a liquid gap for metered liquid delivery.

Another object of the present invention is to provide a physiotherapy device having a switch component rotatably connected to a mounting base, the switch component switchable between a first position connecting a liquid outlet channel to a container bottle and a second position disconnecting the liquid outlet channel from the container bottle.

Another object of the present invention is to provide a physiotherapy device having a microneedle assembly disposed on an outer peripheral surface of a sleeve component for contact with skin during rolling application.

Another object of the present invention is to provide a physiotherapy device having a phototherapy lamp plate disposed in a mounting cavity of a mounting shaft, the sleeve component being at least partially light-transmissive so that therapeutic light is transmitted through the sleeve component toward the skin.

Another object of the present invention is to provide a physiotherapy device having a cooling assembly mounted on an end of a mounting shaft, the cooling assembly including a semiconductor cooling component thermally coupled to a limiting cover having a cold compress surface for contact with skin.

Another object of the present invention is to provide a physiotherapy device in which the circuit board activates or deactivates one or more additional functions, such as phototherapy, cooling, or fan operation, based on whether the motion detection element detects rotation or a stationary condition of the sleeve component.

Another object of the present invention is to provide a physiotherapy device having a sleeve component with a smearing portion and a plurality of liquid guide grooves alternately arranged with a microneedle assembly, the smearing portion in contact with a dispensing ball for distributing liquid to the microneedle assembly during rolling.

Yet another object of the present invention is to provide a physiotherapy device having an infusion channel with a tapering segment connecting a nozzle outlet to an installation port, and a detachable microneedle assembly on the rolling applicator.

### SUMMARY OF THE INVENTION

According to a first aspect of the invention, a physiotherapy device is provided. The physiotherapy device comprising: a mounting base; a container bottle removably attachable to the mounting base; a liquid outlet channel extending from the container bottle to a nozzle outlet formed at a front portion of the mounting base; a rolling applicator assembly mounted at the front portion of the mounting base and configured to rotate relative to the mounting base; a sleeve component forming an outer surface of the rolling applicator assembly, the sleeve component being removable and replaceable independently of the mounting base; wherein the sleeve component comprises a light-transmitting material; at least one phototherapy lamp plate disposed within a mounting shaft of the rolling applicator assembly; wherein the phototherapy lamp plate is configured to emit therapeutic light outward through the sleeve component; wherein the nozzle outlet is positioned adjacent the sleeve component such that skin care product is discharged onto a coating surface of the sleeve component during rotation; and wherein the phototherapy lamp plate is positioned relative to the nozzle outlet such that light emitted outward through the sleeve component passes through skin care product present on the coating surface of the sleeve component during rolling movement.

In one embodiment of the invention, the mounting base further comprises a support portion spaced apart from and located beside the nozzle outlet, the mounting shaft fixed to the support portion and defining a mounting cavity, the phototherapy lamp plate disposed in the mounting cavity, at least a portion of the mounting shaft corresponding to the phototherapy lamp plate being light-transmissive, and the sleeve component detachably mounted outside the mounting shaft.

In one embodiment of the invention, the mounting base further has an installation port and a liquid outlet channel, the installation port located at an end of the mounting base away from the rolling applicator assembly, the liquid outlet channel connecting the nozzle outlet and the installation port.

In one embodiment of the invention, the physiotherapy device further comprising a microneedle assembly detachably mounted on the sleeve component and attached to the coating surface, the microneedle assembly arranged circumferentially around a rotation axis of the rolling applicator assembly.

In one embodiment of the invention, the rolling applicator assembly comprises two sleeve components disposed on opposite ends of the mounting base, the mounting base having an arc surface located between the two rolling applicator assemblies, and the nozzle outlet disposed on the arc surface.

According to a second aspect of the invention, a physiotherapy device is provided. The physiotherapy device comprising: a mounting base; a container bottle removably attachable to the mounting base; a liquid outlet channel extending from the container bottle to a nozzle outlet formed at a front portion of the mounting base; a rolling applicator assembly mounted at the front portion of the mounting base and configured to rotate relative to the mounting base; a sleeve component forming an outer surface of the rolling applicator assembly; a flow regulating mechanism disposed along the liquid outlet channel; wherein the rolling applicator assembly is positioned adjacent the nozzle outlet; and wherein the flow regulating mechanism is operatively associated with the rolling applicator assembly and arranged to permit skin care product flow toward the sleeve component in response to rotational movement of the rolling applicator assembly and to restrict skin care product flow in the absence of rotational movement.

In one embodiment of the invention, the flow regulating mechanism comprises a mechanical actuation structure positioned to be displaced directly by rotation of the rolling applicator assembly.

In one embodiment of the invention, the mechanical actuation structure comprises a ball assembled in an outlet tank of the outlet nozzle, the ball sealed with a groove wall of the outlet tank, and wherein rotation of the rolling applicator assembly drives the ball to rotate such that a liquid gap is formed between the ball and the groove wall for skin care product to pass through.

In one embodiment of the invention, the physiotherapy device further comprising an elastic member located in the outlet tank, one end of the elastic member connected to the groove wall and another end connected to the ball, wherein when the rolling applicator assembly is stationary, the elastic member exerts elastic force on the ball to reseal the ball with the groove wall; and a sealing ring provided at an opening edge of the outlet tank, the ball passing through an opening of the sealing ring and frictionally retained by the sealing ring.

In one embodiment of the invention, the flow regulating mechanism comprises a motion detection element and an electronic control valve configured to regulate skin care product flow based on a detected rotation of the rolling applicator assembly.

In one embodiment of the invention, the motion detection element comprises a photoelectric sensor and a detection unit, the detection unit disposed on the sleeve component and configured to move synchronously with the sleeve component, the photoelectric sensor disposed on the mounting base and comprising an optical transmitter and an optical receiver, and the detection unit configured to rotate between the optical transmitter and the optical receiver to block or conduct transmission of light therebetween.

In one embodiment of the invention, the detection unit comprises a plurality of detection portions evenly spaced along a circumferential direction of the sleeve component, an avoidance gap provided between two adjacent detection portions for light to pass through, and the plurality of detection portions configured to pass through a light channel between the optical transmitter and the optical receiver in turn.

In one embodiment of the invention, the physiotherapy device further comprising a circuit board disposed in the mounting base and electrically connected to the electronic control valve, the circuit board configured to open the electronic control valve based on a first detection signal when the motion detection element detects rotation of the sleeve component, and to close the electronic control valve based on a second detection signal when the motion detection element detects that the sleeve component is stationary; and a stimulation element electrically connected to the circuit board, wherein the circuit board activates the stimulation element based on the first detection signal and deactivates the stimulation element based on the second detection signal.

According to a third aspect of the invention, a physiotherapy device is provided. The physiotherapy device comprising: a mounting base having a liquid outlet channel with a nozzle outlet; a mounting shaft fixed to the mounting base; a sleeve component rotatably mounted on the mounting shaft, the sleeve component having an outer peripheral surface with a microneedle assembly configured for contact with skin, the outer peripheral surface positioned proximate to the nozzle outlet such that liquid from the liquid outlet channel is applied to the outer peripheral surface; a switch component rotatably connected to the mounting base, the switch component configured to connect to a container bottle and to switch between a first position in which the switch component connects the liquid outlet channel to the container bottle and a second position in which the switch component disconnects the liquid outlet channel from the container bottle; and a limiting cover detachably connected to an end of the mounting shaft away from the mounting base, the limiting cover configured to limit axial movement of the sleeve component along the mounting shaft.

In one embodiment of the invention, the switch component is rotatably assembled on a side of the mounting base configured to connect with the container bottle, and the switch component is configured to rotate relative to the mounting base to connect or disconnect the liquid outlet channel and the container bottle.

In one embodiment of the invention, the mounting base is provided with a first insertion tube communicating with the liquid outlet channel, the first insertion tube having a first notch on a tube wall thereof; the switch component is provided with a slot having a bottom wall, a second insertion tube and a liquid inlet cut arranged at intervals on the bottom wall; the first insertion tube disposed in the slot, the second insertion tube rotatably disposed in the first insertion tube and having a second notch on a tube wall thereof; in the first position, the second notch positioned opposite to the first notch; and in the second position, the second notch offset from the first notch such that the tube wall of the second insertion tube blocks the first notch.

In one embodiment of the invention, the outer peripheral surface of the sleeve component is provided with a smearing portion in contact with a ball, the smearing portion spaced apart from the microneedle assembly, and a plurality of liquid guide grooves alternately arranged with the microneedle assembly, at least part of the liquid guide grooves connected to the smearing portion.

In one embodiment of the invention, the mounting shaft defines a mounting cavity, a phototherapy lamp plate disposed in the mounting cavity, at least a portion of the mounting shaft corresponding to the phototherapy lamp plate being light-transmissive, and the sleeve component being at least partially formed of a light-transmitting material.

In one embodiment of the invention, the physiotherapy device further comprising a cooling assembly mounted on the end of the mounting shaft away from the mounting base, the cooling assembly comprising a semiconductor cooling component having a cold end and a hot end disposed opposite to each other, the cold end thermally coupled to the limiting cover to cool the limiting cover, the limiting cover having a cold compress surface configured for contact with skin.

In one embodiment of the invention, the mounting shaft is formed from a heat-insulating material, an outer peripheral wall of the mounting shaft provided with shaft holes connecting the mounting cavity and the sleeve component, an air inlet gap provided between the limiting cover and the mounting shaft communicating with the mounting cavity, and a fan assembly located in the mounting cavity configured to draw air from the air inlet gap and discharge air through the shaft holes.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “skin dispensing and physiotherapy device” refers to any handheld device configured to dispense a liquid skin care product onto skin and to provide one or more physiotherapy functions, such as phototherapy, microneedle therapy, cooling, heating, or massage, during or in conjunction with the dispensing operation.

In the context of the specification, the term “stimulation element” refers broadly to any component, module, or structure configured to apply a therapeutic or cosmetic stimulus to a user's skin or tissue. Stimulation elements may include, but are not limited to, a phototherapy element, a microcurrent element, a Peltier element, a vibrational element, a thermal element, an ultrasonic wave therapy element, a magnetotherapy element, electrical stimulation elements, a galvanic element, a Tens element, an RF element, a pulsed electromagnetic field (PEMF) element, or a combination thereof. The terms “first stimulation element”, “second stimulation element”, and “third stimulation element” are for descriptive purposes and each term includes, but is not limited to, a phototherapy element, a microcurrent element, a Peltier element, a vibrational element, a thermal element, an ultrasonic wave therapy element, a magnetotherapy element, electrical stimulation elements, a galvanic element, a massage element, a Tens element, an RF element, a pulsed electromagnetic field (PEMF) element, or a combination thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, EMS, galvanic current, or equivalent electrical therapy.

In the context of the specification, the term “housing” is intended to cover any casing, enclosure, or structural body that contains or supports components of the device. The housing may include a handle portion, head, or other segments, and may be made from polymeric, metallic, composite, or other suitable materials.

In the context of the specification, the terms “head” or “phototherapy head” or “upper shell” refer to a portion of the device coupled to the housing and configured to emit light toward the skin. The head may include one or more light-transmitting surfaces, optical lenses, or diffusers, and may also support electrodes or other stimulation elements.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “control board or circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the following, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200 nm, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

In an embodiment, the phototherapy unit emits multiple therapeutic wavelengths adapted for skin care and dermatological treatment. Red light (approximately 630-660 nm) penetrates deeply into the skin to stimulate blood circulation, enhance collagen production, and promote skin regeneration and repair. Blue light (around 415-470 nm) exhibits antibacterial properties and is effective in treating acne, minimizing breakouts, and reducing inflammation. Green light (approximately 520-540 nm) helps reduce hyperpigmentation, even skin tone, and soothe sensitive or irritated skin. Yellow light (around 580-600 nm) improves cellular oxygen exchange, enhances lymphatic circulation, and supports detoxification and skin revitalization. Near-infrared light (800-850 nm) penetrates deeper tissue layers to accelerate healing, reduce pain, and alleviate inflammation, thereby supporting overall skin recovery and rejuvenation.

### BRIEF DESCRIPTION OF DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a perspective view of a physiotherapy device, in accordance with an embodiment of the present invention.

FIG. 2 shows a cross-sectional view of the physiotherapy device, in accordance with an embodiment of the present invention.

FIG. 3 shows a cross-sectional view of a phototherapy head of the physiotherapy device, in accordance with an alternate embodiment of the present invention.

FIG. 4 shows a perspective view of the physiotherapy device, showing a pressing actuator, a microneedle assembly, and a container bottle, in accordance with an alternative embodiment of the present invention.

FIG. 5 shows a perspective view of a portion of a physiotherapy device, showing a microneedle assembly, a nozzle, a mounting base, and a switch component, in accordance with an embodiment of the present invention.

FIG. 6 illustrates an exploded view of a portion of a physiotherapy device, showing a microneedle assembly, a nozzle, a mounting base, and a switch component, in accordance with an embodiment of the present invention.

FIG. 7 shows a cross-sectional view of a portion of a physiotherapy device, showing a microneedle assembly, a nozzle, a liquid outlet channel, a mounting base, and a switch component, in accordance with an embodiment of the present invention.

FIG. 8 shows a side perspective view of a portion of a physiotherapy device, showing a microneedle assembly, a nozzle, a mounting base, and a fan assembly, in accordance with an embodiment of the present invention.

FIG. 9 shows a bottom view of a portion of a physiotherapy device, showing a mounting base and an insertion block, in accordance with an embodiment of the present invention.

FIG. 10 shows a top perspective view of a switch component, in accordance with an embodiment of the present invention.

FIG. 11 shows a top perspective view of an engagement between a mounting base and the switch component in an off position, in accordance with an embodiment of the present invention.

FIG. 12 shows a top perspective view of a switch component showing a liquid inlet cut without an insertion boss, in accordance with an embodiment of the present invention.

FIG. 13 shows an enlarged view of part A of FIG. 7, in accordance with an embodiment of the present invention.

FIG. 14 shows a perspective view of an alternate configuration of a physiotherapy device, showing a mounting base, a plurality of nozzles, a switch component, and a container bottle, in accordance with an embodiment of the present invention.

FIG. 15 shows another exploded view of a mounting base, showing a mounting shaft, a sleeve component, and a switch component, in accordance with an embodiment of the present invention.

FIG. 16 shows an enlarged view of part B of FIG. 15, showing a ball arrangement on a nozzle, in accordance with an embodiment of the present invention.

FIG. 17 shows another cross-sectional view of a shell, in accordance with an embodiment of the present invention.

FIG. 18 shows an enlarged view of part C of FIG. 17, showing a ball arrangement on a nozzle, in accordance with an embodiment of the present invention.

FIG. 19 shows another enlarged view of part C of FIG. 17, showing a ball arrangement on a nozzle with a sealing ring, in accordance with an embodiment of the present invention.

FIG. 20 shows a perspective view of an alternate configuration of a physiotherapy device, showing a mounting base, a switch component, a liquid guide groove, a smearing portion, a container bottle, and a nozzle, in accordance with an embodiment of the present invention.

FIG. 21 shows another exploded view of a mounting base, showing a second end wall, a mounting shaft, and a nozzle, in accordance with an embodiment of the present invention.

FIG. 22 shows an enlarged view of part D of FIG. 21, in accordance with an embodiment of the present invention.

FIG. 23 shows an exploded view of a mounting base, showing a mounting shaft, a sleeve component, a control button, and a phototherapy lamp plate, in accordance with an embodiment of the present invention.

FIG. 24 shows an enlarged view of part E of FIG. 23, in accordance with an embodiment of the present invention.

FIG. 25 shows a cross-sectional view of the physiotherapy head, showing a microneedle assembly, a nozzle, an electronic control valve, a liquid outlet channel, and a mounting base, in accordance with an embodiment of the present invention.

FIG. 26 shows an enlarged view of part F of FIG. 25, in accordance with an embodiment of the present invention.

FIG. 27 shows a perspective view of another configuration of a physiotherapy device, showing a support portion, a mounting base, and a container bottle, in accordance with an embodiment of the present invention.

FIG. 28 shows another cross-sectional view of the physiotherapy device, showing a support portion, a mounting base, and a container bottle, in accordance with an embodiment of the present invention.

FIG. 29 shows a perspective view of a mounting base, showing an air inlet and a gas duct, in accordance with an embodiment of the present invention.

### DETAILED DESCRIPTION OF THE INVENTION

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

In some embodiments, a physiotherapy device is provided. The physiotherapy device comprises a physiotherapy head, a mounting base, and a container bottle.

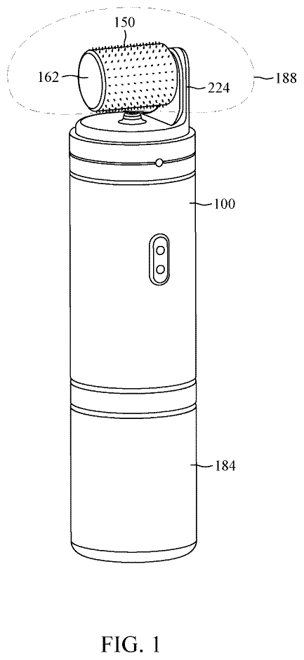

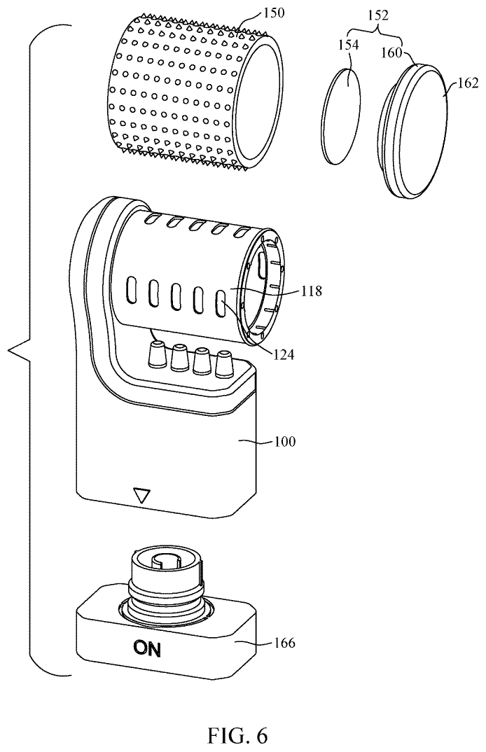

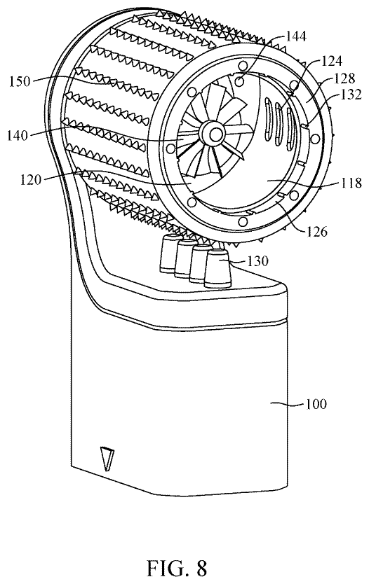

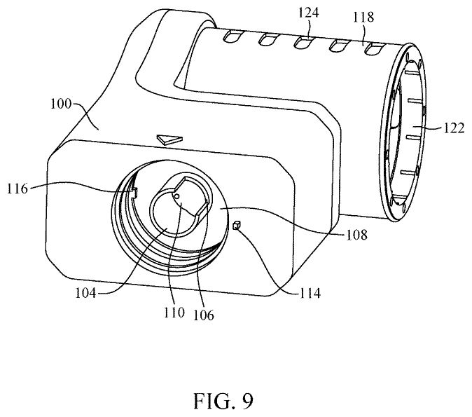

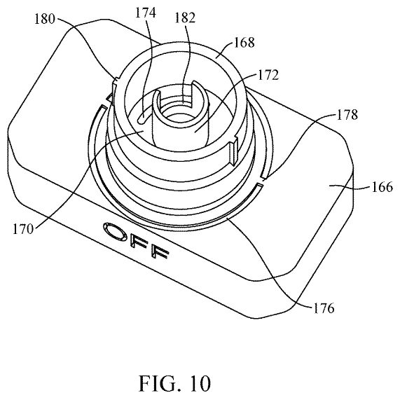

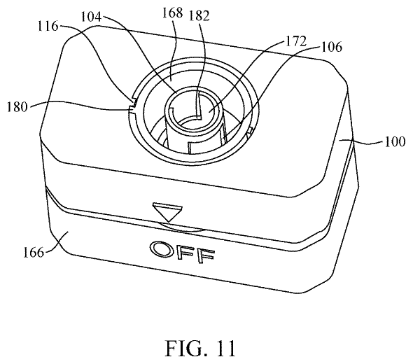

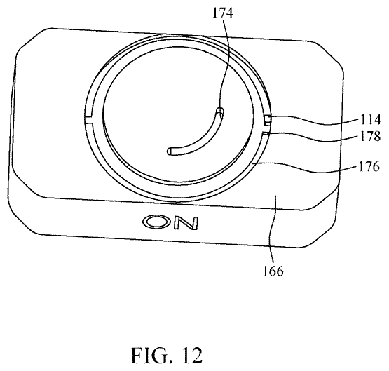

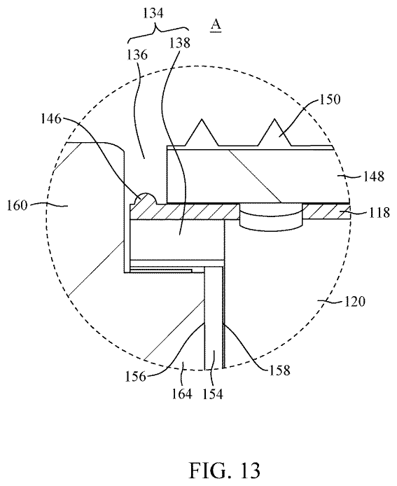

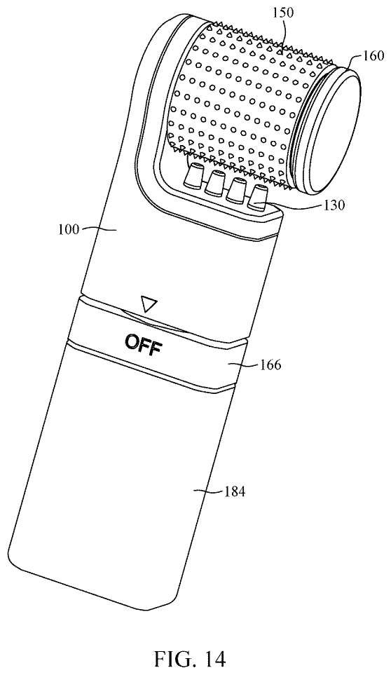

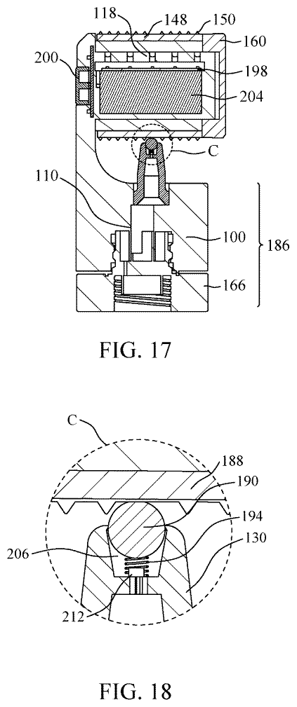

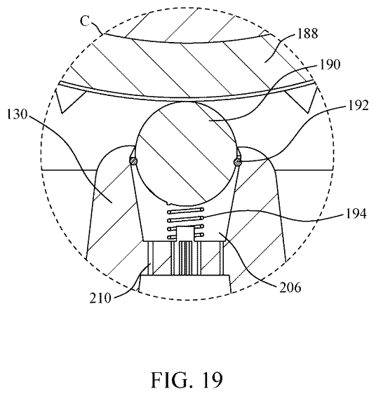

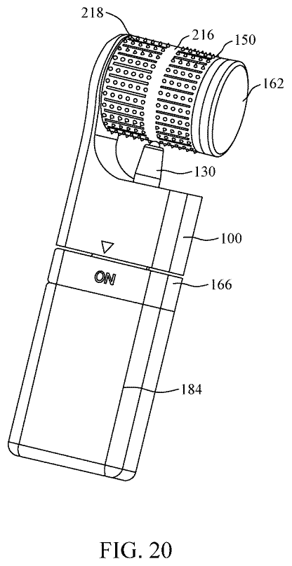

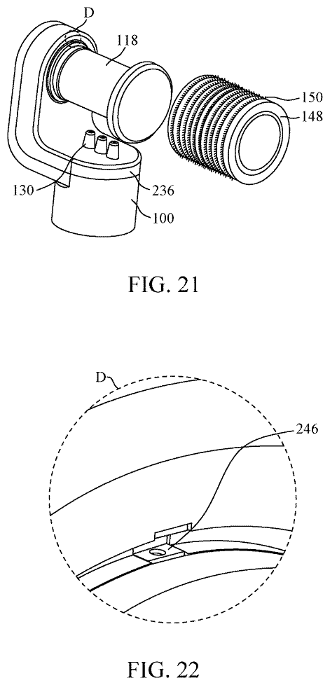

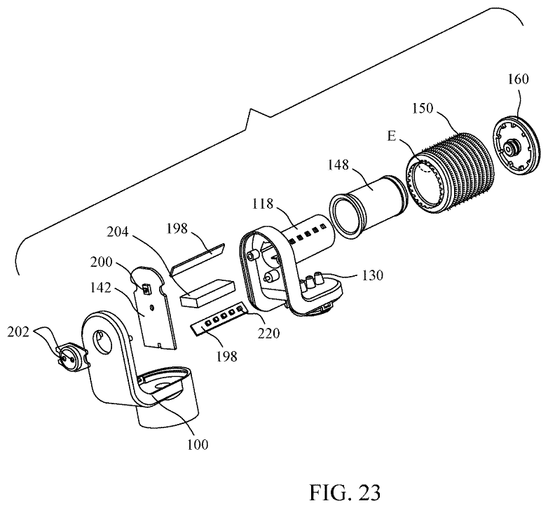

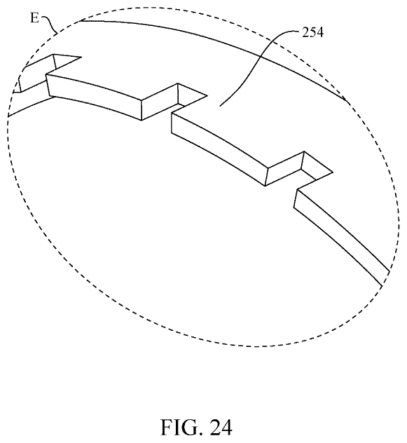

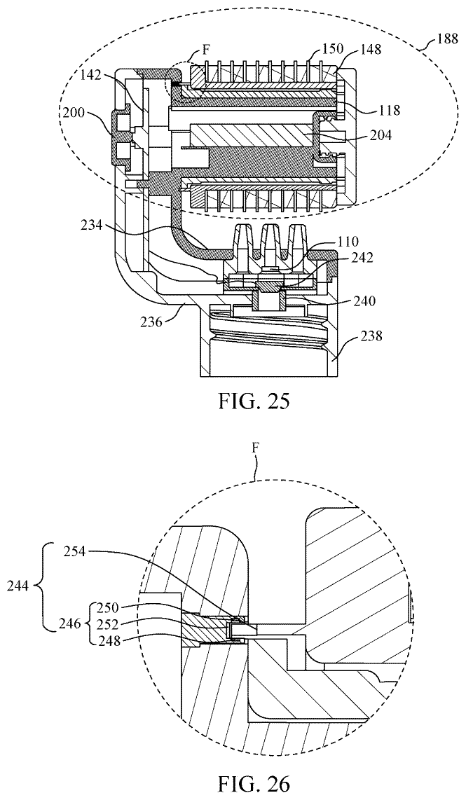

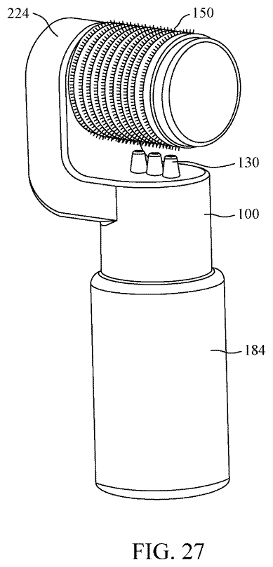

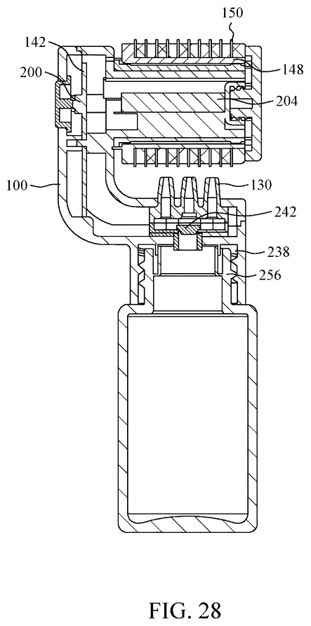

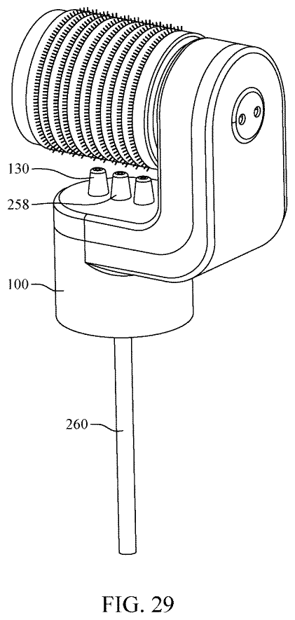

Embodiments of the present invention will now be described in detail with reference to FIGS. 1 to 29, wherein like reference numerals refer to like elements throughout.

Referring to FIGS. 1 to 4, in an embodiment, a physiotherapy device comprises a mounting base 100 that serves as the primary structural body of the device. The mounting base 100 is configured to connect with a container bottle 184 for storing a liquid skin care product or medicinal liquid. The mounting base 100 includes a support portion 224 from which a mounting shaft 118 extends. The mounting shaft 118 is fixed to the support portion 224 of the mounting base 100.

In an embodiment, the support portion 224 can be configured as an L-shaped mounting arm, with a short side connected to the mounting base 100 and a long side extending away from the mounting base 100. The mounting shaft 118 can protrude from the long side of the L-shaped mounting arm in a direction perpendicular to the long side. The space formed between the L-shaped mounting arm and the mounting base 100 can accommodate the sleeve component 148 and the microneedle assembly 150, and the sleeve component 148 can be removed and replaced from the side opposite the mounting arm. In an alternative embodiment, the mounting base 100 can be provided with a U-shaped groove, and the two ends of the mounting shaft 118 can be respectively received in the opposing side walls of the U-shaped groove, so that the mounting shaft 118 is supported at both ends.

In an embodiment, the number of support portions 224 can be one or two. When one support portion 224 is provided, one end of the mounting shaft 118 is fixed to the support portion 224, and the opposite end of the mounting shaft 118 and the sleeve component 148 are cantilevered. When two support portions 224 are provided, one end of the mounting shaft 118 is connected to one of the support portions 224, and the other end is connected to the other support portion 224, so that the mounting shaft 118 is supported at both ends. The mounting arm can be provided with one support portion 224, and in other embodiments, the mounting arm can be provided with two support portions 224 arranged on opposite sides.

In an embodiment, a sleeve component 148 is rotatably assembled on the mounting shaft 118. The sleeve component 148 is configured to rotate around the mounting shaft 118 when the device is rolled across the skin. A bearing can be provided between the mounting shaft 118 and the sleeve component 148 to allow smooth rotation and to reduce wear.

In an embodiment, a shaft sleeve can be fixed on the mounting shaft 118 between the mounting shaft 118 and the sleeve component 148. The sleeve component 148 can be movably sleeved on the shaft sleeve. The outer wall of the shaft sleeve is provided with at least two abutting convex portions that extend circumferentially along the mounting shaft 118 in a ring shape. The outer wall surface of each abutting convex portion is configured to abut against the inner peripheral wall of the sleeve component 148. The at least two abutting convex portions can be arranged at opposite ends of the shaft sleeve, one abutting convex portion being proximate to a flanging portion of the shaft sleeve and the other being proximate to the opposite end of the shaft sleeve. This arrangement can reduce radial jitter of the sleeve component 148 during rotation, thereby improving the rolling stability of the sleeve component 148.

In an embodiment, the sleeve component 148 can be cylindrical, and the coating surface can be a cylindrical surface. In alternative embodiments, the sleeve component 148 can be spherical or ellipsoidal in shape. A spherical or ellipsoidal sleeve component 148 can provide a different contact profile with the skin and can be suited for treating curved or contoured areas of the face or body.

In an embodiment, a liquid outlet channel 110 is disposed within the mounting base 100. The liquid outlet channel 110 connects the exterior of the mounting base 100 to the container bottle 184, providing a fluid path for delivering the skin care product from the container bottle 184 to the sleeve component 148. The liquid outlet channel 110 terminates at a nozzle 130 on the mounting base 100. The nozzle 130 is positioned adjacent to the sleeve component 148 and is configured to deliver liquid from the container bottle 184 to the surface of the sleeve component 148. A nozzle outlet 222 is provided on the nozzle 130 for dispensing liquid onto the sleeve component 148.

In an embodiment, the central axis of the mounting shaft 118 is approximately perpendicular to the opening direction of the nozzle outlet 222. This arrangement allows the coating surface of the sleeve component 148 to smoothly receive the skin care product flowing from the nozzle outlet 222 when the sleeve component 148 is rolling, while avoiding conflict between the rotation direction and the outflow direction of the liquid, and ensuring smooth rolling and coating.

In an embodiment, the coating surface of the sleeve component 148 is spaced apart from the nozzle outlet 222 by a predetermined distance. During use, the skin care product is extruded from the nozzle outlet 222 to a height sufficient to contact the coating surface, thereby avoiding direct contact between the coating surface and the edge portion of the nozzle outlet 222. This arrangement can reduce the risk of contamination of the nozzle outlet 222 by residue on the coating surface. After use, the sleeve component 148 can be rolled on a cleaning device, such as a wet wipe, to clean the coating surface without the cleaning device contacting the nozzle outlet 222.

In an embodiment, a plurality of liquid outlet channels 110 can be provided on the mounting base 100 and fixed at intervals along the axial direction of the sleeve component 148. The plurality of liquid outlet channels 110 can simultaneously dispense liquid onto the microneedle assembly 150 on the sleeve component 148, thereby increasing the dispensing range and ensuring that all microneedles on the sleeve component 148 come into contact with the liquid. A plurality of liquid distribution chambers 112 can be provided within the mounting base 100 along the axial direction of the first insertion tube 104. The liquid outlet channel 110 can connect to a plurality of liquid distribution chambers 112, each liquid distribution chamber 112 corresponding to one of the liquid outlet channels 110. Each liquid distribution chamber 112 can have the same volume, ensuring that each liquid outlet channel 110 dispenses the same amount of liquid, resulting in a uniform distribution of the liquid on the microneedle assembly 150.

In an embodiment, the container bottle 184 is detachably connected to the mounting base 100. The mounting base 100 provides an installation port 226 at the end opposite the physiotherapy head 188 to receive the bottle mouth of the container bottle 184. The installation port 226 can be configured with a standard neck geometry so that commercially available bottles can be installed or replaced. A sealing gasket can be provided to ensure a leak-free connection. The liquid outlet channel 110 leads from the installation port 226 toward the nozzle outlet 222 and can include a tapering segment 228 adjacent to the nozzle outlet 222 to restrict backflow and control dispensing volume.

In an embodiment, the mounting base 100 is provided with a circuit board 142, a battery 204, a charging interface 202, and a control button 200. The circuit board 142 is disposed within the mounting base 100 and is electrically connected to various components of the device. The battery 204 is housed within the mounting base 100 to provide power to the device. The charging interface 202 is electrically connected to the circuit board 142 and to the battery 204. The charging interface 202 can be provided with at least one of a Type-C female port, two conductive shrapnel contacts, two conductive contact pads, or a wireless charging coil for inductive charging. The control button 200 is provided on the exterior of the mounting base 100 for user operation.

In an embodiment, the physiotherapy head 188 is mounted at one end of the mounting base 100 adjacent the nozzle outlet 222 and comprises the sleeve component 148. The sleeve component 148 is cylindrical and defines a coating surface surrounding a rotating axis defined by the mounting shaft 118. The physiotherapy head 188 is removable using a limiting cover 160 to permit cleaning or replacement of the sleeve component 148 and microneedle assembly 150. The limiting cover 160 can be fastened by at least one of a screw engagement into the mounting shaft 118, a snap-catch feature, or a bayonet-card groove arrangement.

In an embodiment, the projections of the limiting cover 160 and the sleeve component 148 onto the central axis of the mounting shaft 118 overlap. The radial end of the limiting cover 160 can act as a physical stop to limit axial movement of the sleeve component 148 and to prevent the sleeve component 148 from detaching from the mounting shaft 118. The limiting cover 160 and the mounting shaft 118 can be detachably connected.

In an embodiment, the limiting cover 160 can be provided independently of the cooling assembly 152. The limiting cover 160 can be provided with a convex column on the side facing the mounting shaft 118. The convex column can be provided with an external thread, and the free end of the mounting shaft 118 can be provided with a threaded hole. The convex column can be screwed into the threaded hole to fix the limiting cover 160 to the mounting shaft 118. The limiting cover 160 can also be provided with strengthening ribs on the side facing the mounting shaft 118 to increase structural rigidity. In this configuration, the limiting cover 160 serves to retain the sleeve component 148 on the mounting shaft 118 without providing a cooling function.

In an embodiment, a portion of the limiting cover 160 located outside the mounting shaft 118 is disc-shaped, with an outer diameter larger than the outer diameter of the sleeve component 148. The outer peripheral wall of that disc-shaped limiting cover 160 is arranged so that it does not protrude radially beyond the microneedles on the microneedle assembly 150, avoiding interference with the user's skin when the microneedles engage the skin.

In an alternative embodiment, the first end wall of the mounting base 100 can be provided with a movable groove. The physiotherapy head 188 can be rotatably assembled in the movable groove, and the physiotherapy head 188 can protrude from the opening of the movable groove from the inside of the movable groove. In this configuration, the sleeve component 148 is received within the movable groove rather than being mounted on an externally projecting mounting shaft 118, providing a more compact form factor.

In an alternative embodiment, the mounting base 100 can be provided with a shaft hole rather than a protruding mounting shaft 118. The physiotherapy head 188 can include a rolling portion and a rotating shaft portion. The rolling portion can be assembled on the shaft hole by the rotating shaft portion, and the rolling portion can rotate synchronously with the rotating shaft portion. The rolling portion and the rotating shaft portion can be fixed by screws, hot injection molding, or can be integrally formed. In this configuration, the rotating shaft portion is part of the physiotherapy head 188 rather than being fixed to the mounting base 100.

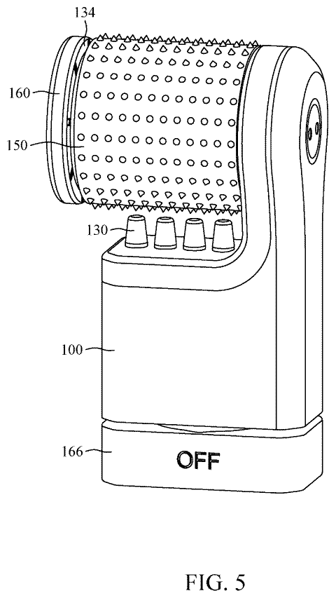

Referring to FIGS. 5 to 8, in an embodiment, the physiotherapy device further includes a microneedle assembly 150 that is disposed on the outer peripheral surface of the sleeve component 148. The microneedle assembly 150 comprises a plurality of microneedles arranged circumferentially around the sleeve component 148 and configured for contact with skin during rolling application. By rolling the sleeve component 148 on the skin, the microneedles puncture the skin to open microchannels so that the skin care product attached to the sleeve component 148 can be guided into the user's skin.

In an embodiment, the microneedle assembly 150 is detachably connected to the sleeve component 148. The microneedle assembly 150 can be formed as a flexible sheet or strip that wraps around the coating surface, as a ring sleeve that is slipped over the sleeve component 148, or as a plurality of discrete microneedle segments that together surround the circumference. The microneedles can be formed from at least one of hydrogel, polymer, silicon, metal, glass, or can be solid, hollow, coated, drug-loaded, or from dissolvable, degradable materials.

In an embodiment, the microneedle assembly 150 can include a cylindrical fixing sleeve into which multiple microneedles are integrally formed and circumferentially spaced on the outer wall of the fixing sleeve. The fixing sleeve and the sleeve component 148 are joined by an interference fit, so the fixing sleeve is fixed to the outer periphery of the sleeve component 148. In one embodiment, the microneedle assembly 150 and the sleeve component 148 are integrally formed. After a microneedle therapy session, the sleeve component 148 can be disassembled and replaced. In other embodiments, the microneedle assembly 150 is implemented as a microneedle patch whose length and width are selected to match the circumference and width of the sleeve component 148. The non-needle side of the patch is adhesive, so the microneedle patch can be affixed to the sleeve component 148 and later peeled off and replaced.

In an embodiment, the microneedle assembly 150 can use a hydrogel material having sufficient softness and tack to adhere to the coating surface without additional adhesive. In an alternative embodiment, the microneedle assembly 150 is provided with an adhesive backing to bond to the coating surface or is mechanically retained by the limiting cover 160. The microneedle assembly 150 is sterile and can be configured as single-use and replaceable or as a reusable component that can be sterilized between uses. The length, density, and tip geometry of the microneedles can be chosen to penetrate the stratum corneum to increase skin permeability and facilitate delivery of active compounds without causing undue discomfort.

In an embodiment, at least part of the microneedle assembly 150 can be formed from a conductive material so that, when the microneedle assembly 150 is energized, the microneedles can deliver microcurrent physiotherapy or radiofrequency physiotherapy to the user's skin. At least one of the sleeve component 148 and the microneedle assembly 150 can be formed from a conductive material selected from metallic conductive materials such as copper, iron, aluminum, gold, silver, or stainless steel, and non-metallic conductive materials such as conductive plastics, conductive rubber, or conductive silicone. A conductive part can be provided on the mounting shaft 118 to electrically connect the circuit board 142 to the sleeve component 148 or the microneedle assembly 150. In one configuration, the conductive part is a conductive ball protruding from the outer wall of the mounting shaft 118, and a conductive groove is provided on the inner wall of the sleeve component 148. When the sleeve component 148 is assembled on the mounting shaft 118, the conductive ball is received in the conductive groove to establish an electrical connection. In another configuration, the conductive part is arranged on the side wall of the support portion 224, and the end of the sleeve component 148 contacts the conductive part when the sleeve component 148 is assembled on the mounting shaft 118.

In an embodiment, the microneedle assembly 150 can be configured so that the microneedles are movable in a direction toward and away from the skin surface. The microneedles can be spring-loaded, pneumatically actuated, or mechanically biased so that, upon contact with the skin, the microneedles retract when a predetermined penetration depth or force threshold is reached. This arrangement can prevent the microneedles from penetrating the skin excessively and can reduce discomfort during treatment. The circuit board 142 can monitor the penetration depth or contact force and can generate an alert to the user when a target depth is reached or when the microneedles should be retracted.

In an embodiment, as an alternative to the microneedle assembly 150, the sleeve component 148 can be provided with a plurality of massage protrusions on the outer wall thereof. The massage protrusions can be arc-shaped, hemispherical, semi-elliptical, or spherical. The massage protrusions can be arranged in circumferential rows around the sleeve component 148 and can be evenly distributed along the axial direction of the sleeve component 148. When the sleeve component 148 is rolled on the skin, the massage protrusions can stimulate the skin to provide massage physiotherapy. The sleeve component 148 with massage protrusions can be interchangeable with the sleeve component 148 carrying the microneedle assembly 150, allowing the user to select the appropriate roller type for the desired treatment.

Referring to FIGS. 5 to 12, in an embodiment, the physiotherapy device further includes a switch component 166 that is rotatably connected to the mounting base 100. The switch component 166 is configured to connect to the container bottle 184 and is positioned on the side of the mounting base 100 opposite to the sleeve component 148. The switch component 166 is operable to switch between a first position in which the switch component 166 connects the liquid outlet channel 110 to the container bottle 184 and a second position in which the switch component 166 disconnects the liquid outlet channel 110 from the container bottle 184.

In an embodiment, the mounting base 100 is provided with a first insertion tube 104 that communicates with the liquid outlet channel 110. A first notch 106 is formed in the wall of the first insertion tube 104. The switch component 166 defines a slot 170. A second insertion tube 172 and a liquid inlet cut 174 are formed on the bottom wall of the slot 170 at separate locations. The liquid inlet cut 174 communicates with the container bottle 184. The liquid outlet channel 110 is connected to the liquid inlet cut 174 via the first notch 106. The first insertion tube 104 is received in the slot 170, and the second insertion tube 172 is rotatably disposed within the first insertion tube 104. The wall of the second insertion tube 172 is provided with a second notch 182.

In the first position, the second notch 182 aligns with the first notch 106 to open a continuous flow path. The liquid outlet channel 110, the second insertion tube 172, the second notch 182, the first notch 106, and the liquid inlet cut 174 are connected in sequence. In the second position, the second notch 182 and the first notch 106 are offset so that the wall of the second insertion tube 172 blocks the first notch 106, and the liquid outlet channel 110 is separated from the liquid inlet cut 174.

In an embodiment, the switch component 166 further includes a rotating portion provided with a limiting groove 176. A first limiting block 178 projects into the limiting groove 176, and an insertion block 114 protrudes from the bottom of the mounting base 100. The insertion block 114 is received in the limiting groove 176. When the rotating portion turns, the first limiting block 178 abuts against the insertion block 114 at both the first and second positions to limit further rotation of the rotating portion.

In an embodiment, the bottom wall of the mounting base 100 defines a rotating groove 108 from which the first insertion tube 104 projects. The peripheral wall of the rotating groove 108 is provided with a second limiting block 116. An insertion boss 168 of the switch component 166 is inserted into the rotating groove 108, and the outer peripheral wall of the insertion boss 168 is provided with a third limiting block 180. When the switch component 166 rotates, the second limiting block 116 abuts against the third limiting block 180 in either the first or second position to limit the rotation.

In an embodiment, the surface of the switch component 166 is marked with the words “ON” and “OFF,” and an arrow is provided on the mounting base 100. When the arrow points to “ON,” the liquid outlet channel 110 is connected to the container bottle 184, and the switch component 166 is in the first position. When the arrow points to “OFF,” the liquid outlet channel 110 is isolated from the container bottle 184, and the switch component 166 is in the second position.

In an embodiment, the switch component 166 is formed with a threaded hole at its bottom into which the opening of the container bottle 184 is screwed. A sealing ring is provided at the connection between the container bottle 184 and the switch component 166 to ensure a sealed joint. This design enables the container bottle 184 to be connected to the mounting base 100 via the switch component 166, and rotating the switch component 166 controls the connection and isolation between the liquid outlet channel 110 and the container bottle 184.

In an alternative embodiment, the switch component 166 can be fixedly connected to the mounting base 100 and slidably connected to the container bottle 184. The switch component 166 can slide along the axis of the container bottle 184 to disengage from or block the opening of the container bottle 184, thereby connecting or isolating the liquid outlet channel 110 from the container bottle 184. In this configuration, the switch component 166 moves axially rather than rotationally to control liquid flow.

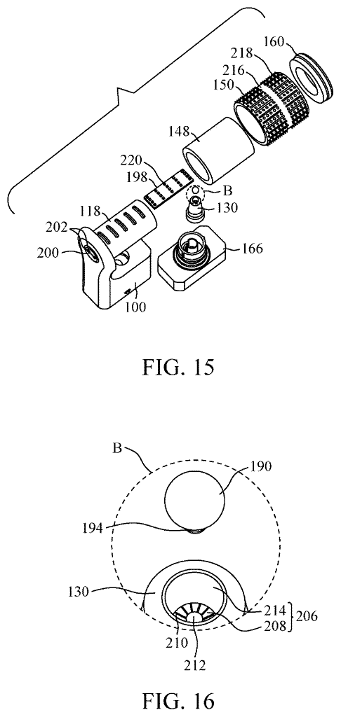

Referring to FIGS. 15 to 20, in an embodiment, the physiotherapy device comprises a shell 186 defining an outlet tank 206 that communicates with the container bottle 184 via the liquid outlet channel 110. A ball 190 is received in the outlet tank 206 and is engaged with the opening edge of the outlet tank 206. A sealing ring 192 is provided at the opening edge of the outlet tank 206 so that the ball 190 passes through the opening of the sealing ring 192 and is frictionally and sealingly retained by the sealing ring 192. The groove wall of the outlet tank 206 can be formed from an elastomeric material so that the ball 190 and the groove wall are in interference contact to achieve sealing.

In an embodiment, the outlet tank 206 comprises a first bottom wall 208 and a first perimeter wall 214. The first perimeter wall 214 facilitates the rolling of the ball 190. The first bottom wall 208 is provided with a nozzle through hole 210 that connects to the liquid outlet channel 110, whereby liquid flows from the container bottle 184, through the switch component 166, the liquid outlet channel 110, and the nozzle through hole 210 into the outlet tank 206.

In an embodiment, an elastic member 194 is arranged in the outlet tank 206 with one end secured to a central positioning feature on the first bottom wall 208 and the other end engaging the ball 190. The elastic member 194 can be at least one of a coil spring, shrapnel, or bullet block. The first bottom wall 208 can include a positioning block 212 on its central axis to locate one end of the elastic member 194 and to define the circumferential distribution of nozzle through holes 210 around the positioning block 212.

In an embodiment, in normal resting conditions, when the ball 190 is at rest, and liquid delivery from the container bottle 184 is stopped, the ball's elastic member 194 biases the ball 190 into sealing contact with the groove wall or sealing ring 192 to prevent gravity-fed leakage when the container is inverted, shaken, or subjected to physical forces. When the sleeve component 148 is driven to rotate relative to the shell 186, frictional contact between the physiotherapy head 188 and the ball 190 causes the ball 190 to rotate against the bias of the elastic member 194. The rotation of the ball 190 momentarily breaks the sealing contact between the ball 190 and the groove wall and forms a narrow, dynamically generated flow gap through which liquid is metered out. The ball 190 thus functions both as a normally-closed valve and as a secondary distributor that receives liquid from the outlet tank 206 and transfers it uniformly to the physiotherapy head 188 during rotation.

In an embodiment, the shell 186 further includes the nozzle 130 projecting on the side adjacent the physiotherapy head 188 so the outlet tank 206 is disposed close to the nozzle 130 and within reach of the sleeve component 148. At least one nozzle 130 is connected to the liquid outlet channel 110 and can be integrally molded with the shell 186 or fixed by hot injection molding or adhesive.

In an embodiment, the nozzle 130 is spaced apart from the sleeve component 148 by a distance sufficient to prevent the microneedle assembly 150 protruding from the outer peripheral surface of the sleeve component 148 from contacting or interfering with the nozzle 130 during rotation of the sleeve component 148. This spacing ensures that the rotation of the sleeve component 148 is not impeded by insufficient clearance between the nozzle 130 and the microneedle assembly 150.

In an embodiment, the physiotherapy head 188 comprises the sleeve component 148 mounted for rotation on the mounting shaft 118 fixed on the shell 186. The sleeve component 148 on its peripheral surface carries a smearing portion 216 in a ring shape and at least one microneedle assembly 150 fixed to the outer surface of the sleeve component 148. The microneedle assembly 150 and the smearing portion 216 are circumferentially spaced so that the ball 190 can transfer liquid onto the smearing portion 216 without obstructing the microneedle assembly 150. A plurality of liquid guide grooves 218 is formed on the peripheral surface of the sleeve component 148 and is arranged alternately with the microneedle assembly 150 so that at least part of each liquid guide groove 218 opens to the smearing portion 216, enabling controlled delivery of the liquid along the microneedle channels into the skin.

In an embodiment, the opening edge of each liquid guide groove 218 can be provided with a bevel. The bevel can guide the liquid on the smearing portion 216 more smoothly into the liquid guide groove 218, and can also facilitate guiding the liquid from the liquid guide groove 218 toward the microneedle assembly 150 during rolling.

In an embodiment, the sleeve component 148 can include a plurality of smearing portions 216 spaced along the peripheral surface, each smearing portion 216 being arranged between adjacent microneedle assemblies 150 and fluidly associated with at least one liquid guide groove 218. Each smearing portion 216 is configured to receive liquid from the ball 190 and distribute the liquid into adjacent liquid guide grooves 218 for delivery toward the microneedle assemblies 150.

In an embodiment, the outer surface of the smearing portion 216 can be provided with anti-slip patterns or bumps to increase the friction between the sleeve component 148 and the ball 190. The increased friction can cause the ball 190 to rotate more reliably when the sleeve component 148 rotates, thereby improving the consistency of liquid application to the smearing portion 216 through the ball 190.

In an embodiment, the physiotherapy device can incorporate more than one nozzle 130, each nozzle 130 being associated with the respective outlet tank 206 and the ball 190 arranged with a sealing ring 192 and the elastic member 194. The plurality of balls 190 can be circumferentially spaced around the physiotherapy head 188 so that, upon rotation of the sleeve component 148, each ball 190 independently forms a metered flow gap and transfers liquid from the respective outlet tank 206 to the rotating surface.

In an embodiment, both the plurality of balls 190 and the plurality of smearing portions 216 can be used in combination to achieve multi-point metered dispensing and uniform application while maintaining the normally closed sealing function provided by the sealing ring 192 and elastic member 194.

In an alternative embodiment, a liquid outlet channel can be provided within the mounting shaft 118, and a plurality of capillaries connected to the liquid outlet channel can be provided within the sleeve component 148. The capillaries can extend from the inner peripheral wall of the sleeve component 148 to the outer surface of the sleeve component 148. In use, liquid from the container bottle 184 can be introduced into the capillaries via the liquid outlet channel in the mounting shaft 118, and the capillary openings on the outer surface of the sleeve component 148 can output the liquid directly onto the outer surface of the sleeve component 148 for application to the skin.

Referring to FIGS. 21 to 28, in an embodiment, the physiotherapy device comprises a mounting shaft 118 fixed to the mounting base 100. The sleeve component 148 is rotatably assembled on the mounting shaft 118 and carries at least a removable microneedle assembly 150. The sleeve component 148 is provided with at least one detection unit 254 disposed circumferentially. The sleeve component 148 is secured to the mounting shaft 118 by a retention clip or screw and is removable for cleaning or replacement. The microneedle assembly 150 is provided as a cartridge that is releasably attached to the outer surface of the sleeve component 148 so that microneedles can be replaced independently of the roller core.

In an embodiment, the mounting base 100 comprises a first end wall 234 and a second end wall 236. The second end wall 236 is arranged for detachable connection to the container bottle 184. A first connecting portion 238 on the second end wall 236 is configured to mate with a second connecting portion 256 on the container bottle 184 by at least one of a thread, bayonet, or snap buckle to permit easy removal and replacement of the container bottle 184.

In an embodiment, the nozzle 130 is formed within the mounting base 100 and contains the liquid outlet channel 110, which passes from an inlet section 240 sealed to the container bottle 184 at the second end wall 236 and terminates at the nozzle through hole 210 on the outer surface of the first end wall 234.

In an embodiment, an electronic control valve 242 is positioned inside the nozzle 130 and is in-line with the liquid outlet channel 110 so that it can block or permit fluid flow from the inlet section 240 to the nozzle through hole 210. The electronic control valve 242 can be implemented as a miniature normally-closed solenoid valve comprising a valve body, valve seat, plunger or armature, return spring, and solenoid coil. The solenoid coil has electrical leads routed to the circuit board 142 through a sealed threading hole. The coil is driven by a power transistor or MOSFET on the circuit board 142 under microprocessor control.

In an embodiment, when the circuit board 142 receives a first detection signal, it energizes the MOSFET that supplies current to the solenoid coil. The energized coil retracts the plunger against the return spring, opening the valve seat and creating a flow path through the liquid outlet channel 110 to the nozzle through hole 210. When the circuit board 142 receives a second detection signal or a timeout or a fault condition occurs, it withdraws coil current so the return spring pushes the plunger back against the valve seat, closing the liquid outlet channel 110.

In an embodiment, a motion detection element 244 is incorporated into the device structure to detect rotation of the sleeve component 148 relative to the mounting shaft 118. The motion detection element 244 includes a photoelectric sensor 246 comprising an optical transmitter 248 and an optical receiver 250 disposed on opposing walls of a detection slot 252 in the mounting base 100 adjacent to the mounting shaft 118. The photoelectric sensor 246 resides on the groove wall of a guide rail groove, and the detection slot 252 is sized and oriented so that ambient light is excluded and a stable light channel is formed between the optical transmitter 248 and the optical receiver 250.

In an embodiment, a detection unit 254 is positioned on the sleeve component 148 and is configured to pass through the detection slot 252 during rotation, thereby blocking or conducting light transmission between the optical transmitter 248 and the optical receiver 250 to generate detection signals that are processed by the circuit board 142 to control the electronic control valve 242 and other device functions. The detection unit 254 comprises a plurality of detection portions that are evenly spaced along the circumference, with avoidance gaps provided between adjacent detection portions to allow light to pass through.

In an embodiment, upon rotation, each passage of the detection unit 254 through the optical channel produces a pulse at the optical receiver 250, which is transmitted to the circuit board 142. The circuit board 142 counts pulses in a preset time window and compares the counted pulses to a preset threshold. When the counted pulses within the preset time meet or exceed the threshold, the circuit board 142 generates a first detection signal and, in response, drives the electronic control valve 242 to open the liquid outlet channel 110 and simultaneously enables physiotherapy head 188 functions such as energizing lamp beads 220 and activating a vibrating member. When the pulse count is below the threshold within the preset time, the circuit board 142 outputs a second detection signal to de-energize the electronic control valve 242 and to stop the physiotherapy head 188 functions.

In an embodiment, the motion detection element 244 can optionally be at least one of an accelerometer, a Hall sensor, or a rotary encoder. The photoelectric sensor 246 can be implemented as a through-beam sensor so that passage of the detection unit 254 blocks the beam and generates a falling edge at the optical receiver 250, or as a reflective sensor with emitter and receiver on the same side. The optical transmitter 248 can be a modulated IR LED, and the optical receiver 250 can be a matched photodiode with a bandpass amplifier. Modulation and synchronous detection can reduce false pulses from ambient light.

In an embodiment, the circuit board 142 implements de-bouncing, pulse width discrimination, and majority voting across multiple detection units 254 to increase reliability. The circuit board 142 also implements a hysteresis timer so that brief pauses do not immediately cut dispensing, while longer pauses that fall below the threshold for the preset time do cause valve closure. Safety interlocks in the firmware prevent valve opening unless the motion detection confirms motion and also enforce maximum continuous dispense durations and minimum time between dispense bursts to prevent over-dispensing and to conserve the battery 204.

In an embodiment, the electronic control valve 242 is mounted with an O-ring and threaded retention in the nozzle 130 to permit removal for service. The valve seat and plunger materials are selected to resist fouling from skin care products.

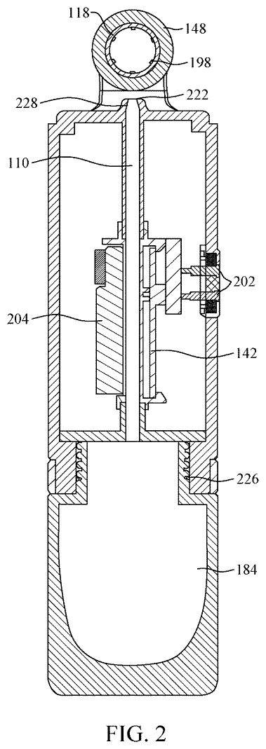

Referring to FIGS. 2, 3, 15, 17, and 23, in an embodiment, the mounting shaft 118 defines a mounting cavity 120. At least one phototherapy lamp plate 198 is installed in the mounting cavity 120 so that the phototherapy lamp plate 198 is fixed relative to the mounting base 100 and does not rotate with the sleeve component 148. The region of the mounting shaft 118 corresponding to the phototherapy lamp plate 198 can be formed from a light-transmissive material and can be provided with shaft holes 124 to allow light to be emitted from the mounting cavity 120 through the mounting shaft 118 toward the coating surface.

In an embodiment, the phototherapy lamp plate 198 carries a plurality of lamp beads 220, which can be LEDs or other light sources and which are selectable to emit one or more wavelengths depending on the therapeutic regime. The user can select wavelengths using the control button 200, via the circuit board 142. The phototherapy lamp plate 198, control button 200, charging interface 202, and battery 204 are electrically connected to the circuit board 142 to provide power, control, and charging functions.

In an embodiment, the portion of the sleeve component 148 corresponding to the coating surface is at least partially light-transmissive, formed from at least one of a transparent or translucent polymer or provided with light-emitting apertures so that light emitted from the phototherapy lamp plate 198 can be emitted through the sleeve component 148 and incident on the skin while the sleeve component 148 is rolled, thereby enabling simultaneous application of cosmetics and light therapy.

In an embodiment, the phototherapy lamp plate 198 can be a flexible lamp plate arranged in a spiral configuration within the mounting shaft 118 to irradiate in the circumferential direction of the mounting shaft 118. The spiral arrangement can increase the coverage area of the phototherapy light around the full circumference of the sleeve component 148, reducing the number of lamp beads 220 needed while maintaining uniform light distribution. A plurality of phototherapy lamp plates 198 can be provided and evenly spaced along the circumferential direction of the mounting shaft 118.

In an embodiment, the phototherapy lamp plate 198 can be mounted on the support portion 224 or the mounting base 100 rather than within the mounting shaft 118. The support portion 224 or the mounting base 100 can be configured as a light-transmitting structure so that the phototherapy lamp plate 198 can illuminate the skin from the mounting base 100 side. In this configuration, the phototherapy lamp plate 198 can be used independently for standalone phototherapy without rolling the sleeve component 148, thereby increasing the versatility of the device.

In an embodiment, the phototherapy lamp plate 198 can be fixed or embedded in the sleeve component 148 so that the phototherapy lamp plate 198 rotates with the sleeve component 148. In that configuration, a collector ring or slip ring or a wireless coil arrangement is provided between the sleeve component 148 and the support portion 224 to maintain an electrical connection between the rotating phototherapy lamp plate 198 and the circuit board 142.

In an embodiment, a light transmission sleeve is fixed around the mounting shaft 118 and sealingly covers the shaft holes 124 so that light emitted by the lamp beads 220 is transmitted through the light transmission sleeve into the sleeve component 148. The light transmission sleeve is fixed to the mounting shaft 118 by adhesives or overmolding and thereby prevents liquid ingress into the mounting cavity 120 while allowing optical output.

In an embodiment, the phototherapy lamp plate 198 is electrically connected to the circuit board 142, which controls lamp on-off, pulse parameters, and power for the phototherapy lamp plate 198 supplied by the battery 204 under the management of the circuit board 142. The circuit board 142 includes control firmware for timed irradiation, intensity adjustment, and safety interlocks that prevent phototherapy lamp plate 198 operation unless the sleeve component 148 is properly installed and the limiting cover 160 is engaged.

Referring to FIGS. 5 to 8 and 13, in an embodiment, a cooling assembly 152 is mounted on the end of the mounting shaft 118 away from the mounting base 100. The cooling assembly 152 includes a semiconductor cooling component 154 and a limiting cover 160. The semiconductor cooling component 154 has a cold end 156 and a hot end 158 disposed opposite one another. The cold end 156 is thermally coupled to the limiting cover 160 to cool the limiting cover 160. The limiting cover 160 provides a cold compress surface 162 for skin contact.

In an embodiment, after the limiting cover 160 has absorbed and stored cold energy, the cold compress surface 162 is applied to the treatment area to cool the skin, reduce swelling, and relieve pain. This integrated cooling arrangement eliminates the need for separate ice packs or fans, simplifying the cold-compress step and improving the user experience.

In an embodiment, the limiting cover 160 is formed from a non-phase-change material whose specific heat capacity is greater than or equal to 0.4 kJ/(kg·K). The non-phase-change material can be selected from at least one of aluminum, stainless steel, aluminum alloys, marble, granite, or ceramics. The limiting cover 160 can be a single material or a mixture.

In an embodiment, the limiting cover 160 comprises a heat-conducting container filled with polyethylene glycol (PEG), with the PEG contained within that container, and the cold compress surface 162 located on the side of the container away from the semiconductor cooling component 154. During cold-storage, the PEG is cooled to its phase-change point and solidifies. When the cold compress surface 162 is later applied to the skin, the heat flow from the skin melts the PEG, which absorbs latent heat during melting and thereby provides sustained cooling.

In an embodiment, a connecting convex shaft 164 projects from the side of the limiting cover 160 facing the mounting shaft 118. The radius of the connecting convex shaft 164 is smaller than the radius of the mounting shaft 118. The connecting convex shaft 164 is detachably inserted into a shaft opening 122 of the mounting cavity 120, and the end of the connecting convex shaft 164 abuts the cold end 156. The connection between the connecting convex shaft 164 and the mounting shaft 118 can be an interference fit or a threaded connection.

In an embodiment, the shaft opening 122 of the mounting cavity 120 can be provided with a plurality of elastic protrusions 132 that extend axially and are evenly spaced around the circumference. The connecting convex shaft 164 is interference-fit into the shaft opening 122. Alternatively, the outer peripheral wall of the connecting convex shaft 164 can be externally threaded, and the shaft opening 122 can be a threaded hole so that the connecting convex shaft 164 can be screwed into the shaft opening 122.

In an embodiment, when the cooling assembly 152 is mounted on the end of the mounting shaft 118, the sleeve component 148 can be replaced by first removing the limiting cover 160 and the semiconductor cooling component 154 from the mounting shaft 118, then sliding the sleeve component 148 along the axial direction of the mounting shaft 118 to remove it from the mounting shaft 118. A replacement sleeve component 148 with a new microneedle assembly 150 can then be slid onto the mounting shaft 118, and the limiting cover 160 and the semiconductor cooling component 154 can be reinstalled onto the mounting shaft 118. When the device is not in use, the sleeve component 148 can be removed from the mounting shaft 118 to avoid soiling or damaging the microneedle assembly 150 during storage.

In an embodiment, the mounting shaft 118 is formed with the mounting cavity 120 and the shaft opening 122 that communicates the mounting cavity 120 with the outside. The semiconductor cooling component 154 is mounted at the shaft opening 122 and closes at least a portion of that shaft opening 122. The hot end 158 of the semiconductor cooling component 154 is located within the mounting cavity 120 and heats the mounting cavity 120. The limiting cover 160 is mounted at the end of the mounting shaft 118 away from the mounting base 100 and is at least partly located outside the mounting cavity 120 and is in close thermal contact with the cold end 156.

In an embodiment, the hot end 158 of the semiconductor cooling component 154 faces into the mounting cavity 120 so that the heat emitted by the hot end 158 can be transferred to the mounting shaft 118 and the sleeve component 148. The heat radiated from the sleeve component 148 benefits pore opening and circulation. A gap or an insulation structure can be provided between the limiting cover 160 and the sleeve component 148 to prevent direct thermal shorting between cold and heat. For example, an arc-shaped protrusion 146 protrudes from the mounting shaft 118, formed of an insulating material and extending in an annular fashion along the axial direction of the mounting shaft 118. The arc-shaped protrusion 146 and the side of the mounting base 100 can also limit axial movement of the sleeve component 148.

In an embodiment, the microneedle assembly 150 is detachably connected to the sleeve component 148.

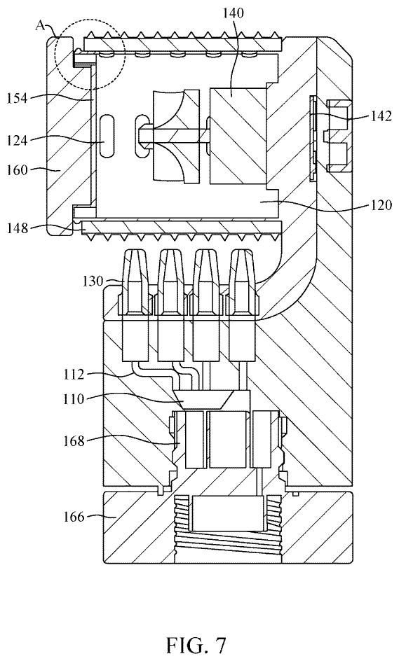

Referring to FIGS. 7, 8, and 13, in an embodiment, the mounting shaft 118 can be formed from a heat-insulating material, and its outer peripheral wall is provided with shaft holes 124 that connect the mounting cavity 120 with the sleeve component 148. When the semiconductor cooling component 154 is operating, the heat-insulating mounting shaft 118 resists absorbing and conducting heat, which reduces heat conduction from the mounting cavity 120 to the limiting cover 160 via the mounting shaft 118 and thereby enhances the cooling effect. At the same time, the shaft holes 124 allow heat produced by the hot end 158 to be guided through the shaft holes 124 to the inner peripheral wall of the sleeve component 148 so the sleeve component 148 can absorb that heat.

In an embodiment, the semiconductor cooling component 154 closes the shaft opening 122, and a breathable air inlet gap 134 is provided between the limiting cover 160 and the mounting shaft 118 that communicates with the mounting cavity 120. The sleeve component 148 is provided with an air-outlet gap between itself and the mounting shaft 118 that communicates with the shaft holes 124 and the exterior.

In an embodiment, a fan assembly 140 located in the mounting cavity 120 draws air from the air inlet gap 134 and discharges it through the shaft holes 124. During this airflow process, the heat generated by the hot end 158 can be expelled through the shaft holes 124. The fan assembly 140 provides forced airflow, accelerating heat transfer, improving heat conduction efficiency, reducing heat accumulation inside the mounting cavity 120, and ensuring stable operation of the semiconductor cooling component 154.

In an embodiment, the fan assembly 140 can be implemented as an exhaust fan or as a piezoelectric ceramic fan. When an exhaust fan is used, it can be installed at the end of the mounting cavity 120 opposite the semiconductor cooling component 154, drawing air from outside into the mounting cavity 120, creating negative pressure and discharging airflow via the shaft holes 124.

In an embodiment, the air inlet gap 134 comprises a first air inlet section 136 and a second air inlet section that are connected. The first air inlet section 136 is located in the space between the ends of the limiting cover 160 and the sleeve component 148. The second air inlet section is formed by one or more air inlet holes 138 located at the end of the mounting shaft 118. These air inlet holes 138 connect the mounting cavity 120 to the first air inlet section 136.

In an embodiment, at the shaft opening 122 of the mounting cavity 120, a protruding ring 128 is provided on the inner peripheral wall of the mounting cavity 120. A baffle plate 126 projects from the protruding ring 128 toward the axis of the mounting cavity 120. The baffle plate 126 is annular, and the semiconductor cooling component 154 is fixed to that baffle plate 126. The second air inlet section is formed by one or more air inlet holes 138 that penetrate the protruding ring 128 and extend axially along the mounting shaft 118.

In an embodiment, the circuit board 142 is mounted on the mounting base 100 and is located outside the mounting cavity 120 to reduce heat transfer from the mounting cavity 120 to the circuit board 142. The mounting base 100 is provided with a wiring hole 144 that communicates with the mounting cavity 120 and through which power and signal lines can pass. The circuit board 142 is electrically connected to the semiconductor cooling component 154 and the fan assembly 140 via wires routed through the wiring hole 144.

Referring to FIG. 29, in an embodiment, in a simple passive gravity-capillary-feed arrangement, the container bottle 184 is mounted below or behind the mounting base 100 so that the nozzle 130 faces the physiotherapy head 188 to be wetted. An air inlet 258 is provided adjacent to the nozzle 130, and a straight gas duct 260 runs from that air inlet 258 into the headspace of the container bottle 184. The air inlet 258 is an open vent that can be fitted with a dust screen. The gas duct 260 is a straight tube terminating above the liquid level. In operation, liquid is delivered by gravity or capillary action from the container bottle 184 through the nozzle 130 as the physiotherapy head 188 rolls, while ambient air enters through the air inlet 258 and travels down the gas duct 260 to replace the displaced volume and prevent a vacuum, producing a steady, self-regulating flow.

In an alternative embodiment, the air inlet 258, gas duct 260, nozzle 130, and container bottle 184 can be configured as a pressure-pulse dispensing system. The air inlet 258 can be configured as a short-stroke actuating interface that, when pressed or depressed by the user, introduces a brief pulse of air pressure into the gas duct 260. The gas duct 260 can be configured as a smooth-bore pressure duct with one-way protection so that the pressure pulse travels from the air inlet 258 into the headspace of the container bottle 184 without backflow. The container bottle 184 can be a rigid reservoir having a small headspace sized to accept the brief pressure pulse, so that the pressure pulse displaces a metered volume of liquid from the container bottle 184 through the liquid outlet channel 110 and toward the nozzle 130. The nozzle 130 can be provided with a check valve or check functionality to prevent dribble or backflow of liquid after the pressure pulse subsides. In this configuration, each actuation of the air inlet 258 delivers a controlled dose of liquid to the sleeve component 148 without requiring an electronic control valve or battery power.

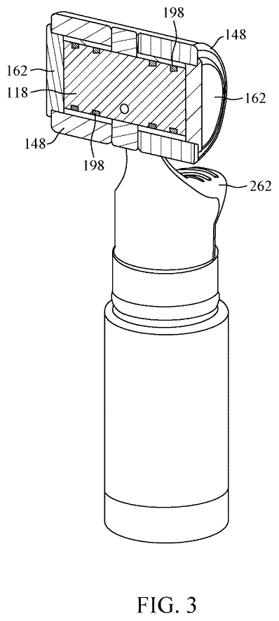

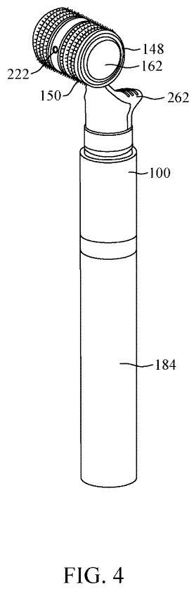

Referring to FIGS. 3 and 4, in an embodiment, a pressing actuator 262 is provided on the exterior of the mounting base 100 and is connected to at least one of an internal pump or one-way valve arrangement in the liquid outlet channel 110. Actuation of the pressing actuator 262 mechanically compresses the pump or opens a valve so that cosmetic fluid is drawn from the container bottle 184 through the installation port 226, the liquid outlet channel 110, and is extruded from the nozzle outlet 222 onto the smearing portion 216 or directly into the gap between the coating surfaces. The pressing actuator 262 is shaped and positioned for thumb or palm operation, facilitating ergonomic pressing and can provide tactile feedback and a click action to indicate a measured dose.

In an embodiment, two physiotherapy heads 188 are provided on opposite sides of one end of the mounting base 100, the two sleeve components 148 being arranged coaxially and forming two opposing coating surfaces with the smearing portion 216 of the mounting base 100 located between them. The nozzle 130 can protrude from the smearing portion 216 and incorporate the nozzle outlet 222 such that the nozzle outlet 222 is arranged at the midline between the opposing coating surfaces.

In an embodiment, two sets of physiotherapy heads 188 can be provided on a single mounting shaft 118. The two physiotherapy heads 188 can be arranged adjacent to each other on the mounting shaft 118 and can rotate independently or synchronously. When replacing the physiotherapy heads 188, the mounting shaft 118 can first be removed from the support portion 224, and then the individual physiotherapy heads 188 can be removed from the mounting shaft 118. The mounting shaft 118 can be fixed to the support portion 224 by a thread or buckle connection to facilitate removal and reinstallation.

In an embodiment, the physiotherapy device is used together with the container bottle 184. The container bottle 184 is detachably connected to the switch component 166 that controls the connection between the liquid outlet channel 110 and the container bottle 184. The container bottle 184 stores at least one of liquid, gas, mixture, serum, cream, or medicinal liquid.

In an embodiment, the sleeve component 148 can be provided in multiple interchangeable types, for example, microneedle roller, massage bump roller, or smearing-only roller, which are fixed to the mounting shaft 118 by interference fit, screw, snap, or integrated molding. A removable cover cap is provided to cover the sleeve component 148 and microneedle assembly 150 when the device is not in use.

In an embodiment, a method of use is provided for the physiotherapy device. With the physiotherapy device powered by the battery 204 and the circuit board 142 enabled, the user places the physiotherapy head 188 on the skin and begins rolling the sleeve component 148. Detection units 254 on the sleeve component 148 pass through the detection slot 252, producing pulses on the photoelectric sensor 246, which are counted by the microcontroller on the circuit board 142. When the pulse count within a preset window meets the threshold, the circuit board 142 outputs a first detection signal that concurrently energizes the electronic control valve 242 to open, energizes the lamp beads 220 on the phototherapy lamp plate 198 to emit selected wavelengths, and activates the fan assembly 140 and/or the cooling assembly 152 if cooling mode is selected. When pulses fall below the threshold, the circuit board 142 sends a second detection signal to close the electronic control valve 242 and stop phototherapy and cooling.

In an embodiment, the circuit board 142 includes a Bluetooth Low Energy radio and a secure firmware stack that exposes physiotherapy device control and telemetry to a companion app. Via the app, the user can select mode, set phototherapy wavelength and intensity, dispense pulse width and per-roll dosing, cooling target temperature and fan speed, view battery 204 state and usage logs, and trigger firmware update and diagnostics.

In an embodiment, the physiotherapy device provides different modes controlled through the circuit board 142, which can include a manual button mode where the control button 200 toggles the phototherapy lamp plate 198, the cooling assembly 152, and the electronic control valve 242 directly; a motion-sensed auto mode where the detection unit 254 and photoelectric sensor 246 automatically open the electronic control valve 242 and start physiotherapy head 188 electronics while the sleeve component 148 is rotating above a threshold; a timed-pulsed mode where the physiotherapy device uses the circuit board 142 timer to apply phototherapy pulses, dispense pulses, or cooling cycles with programmable on-off durations; and an app profile mode where the app issues complex preferred sequences of stimulations and therapies.

In an embodiment, each function can be independently enabled or disabled by a combination of the switch component 166 via mechanical open-close function, the control button 200 for local user input, and app commands. The circuit board 142 arbitrates conflicts and enforces safety interlocks. Profiles and user preferences can be saved locally and optionally synchronized to cloud storage.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A physiotherapy device comprising:
a mounting base;
a container bottle removably attachable to the mounting base;
a liquid outlet channel extending from the container bottle to a nozzle outlet formed at a front portion of the mounting base;
a rolling applicator assembly mounted at the front portion of the mounting base and configured to rotate relative to the mounting base;
a sleeve component forming an outer surface of the rolling applicator assembly;
wherein the sleeve component comprises a light-transmitting material;
at least one phototherapy lamp plate disposed within the rolling applicator assembly;
wherein the phototherapy lamp plate is configured to emit therapeutic light outward through the sleeve component;
wherein the nozzle outlet is positioned adjacent the sleeve component such that skin care product is discharged onto a coating surface of the sleeve component during rotation.

2. The physiotherapy device of claim 1, wherein the sleeve component is removable and replaceable independent of the mounting base.

3. The physiotherapy device of claim 1, wherein the phototherapy lamp plate is positioned relative to the nozzle outlet such that light emitted outward through the sleeve component passes through the skin care product present on the coating surface of the sleeve component during rolling movement.

4. The physiotherapy device of claim 1, wherein the mounting base further comprises a support portion spaced apart from and located beside the nozzle outlet, a mounting shaft fixed to the support portion and defining a mounting cavity, the phototherapy lamp plate disposed in the mounting cavity, at least a portion of the mounting shaft corresponding to the phototherapy lamp plate being light-transmissive, and the sleeve component detachably mounted outside the mounting shaft.

5. The physiotherapy device of claim 1, further comprising a microneedle assembly detachably mounted on the sleeve component and attached to the coating surface, the microneedle assembly arranged circumferentially around a rotation axis of the rolling applicator assembly.

6. The physiotherapy device of claim 1, wherein the rolling applicator assembly comprises two sleeve components disposed on opposite ends of the mounting base, the mounting base having an arc surface located between the two rolling applicator assemblies, and the nozzle outlet disposed on the arc surface.

7. A physiotherapy device comprising:
a mounting base;
a container bottle removably attachable to the mounting base;
a liquid outlet channel extending from the container bottle to a nozzle outlet formed at a front portion of the mounting base;
a rolling applicator assembly mounted at the front portion of the mounting base and configured to rotate relative to the mounting base;
a sleeve component forming an outer surface of the rolling applicator assembly;
a flow-regulating mechanism disposed along the liquid outlet channel;
wherein the rolling applicator assembly is positioned adjacent to the nozzle outlet; and
wherein the flow regulating mechanism is operatively associated with the rolling applicator assembly and arranged to permit skin care product flow toward the sleeve component in response to rotational movement of the rolling applicator assembly and to restrict skin care product flow in the absence of rotational movement.

8. The physiotherapy device of claim 7, wherein the flow-regulating mechanism comprises a mechanical actuation structure positioned to be displaced directly by rotation of the rolling applicator assembly.

9. The physiotherapy device of claim 8, wherein the mechanical actuation structure comprises a ball assembled in an outlet tank of the nozzle outlet, the ball sealed with a groove wall of the outlet tank, and wherein rotation of the rolling applicator assembly drives the ball to rotate such that a liquid gap is formed between the ball and the groove wall for skin care product to pass through.

10. The physiotherapy device of claim 9, further comprising an elastic member located in the outlet tank, one end of the elastic member connected to the groove wall and another end connected to the ball, wherein when the rolling applicator assembly is stationary, the elastic member exerts elastic force on the ball to reseal the ball with the groove wall; and a sealing ring provided at an opening edge of the outlet tank, the ball passing through an opening of the sealing ring and frictionally retained by the sealing ring.

11. The physiotherapy device of claim 7, wherein the flow-regulating mechanism comprises a motion detection element and an electronic control valve configured to regulate skin care product flow based on a detected rotation of the rolling applicator assembly.

12. The physiotherapy device of claim 11, wherein the motion detection element comprises a photoelectric sensor and a detection unit, the detection unit disposed on the sleeve component and configured to move synchronously with the sleeve component, the photoelectric sensor disposed on the mounting base and comprising an optical transmitter and an optical receiver, and the detection unit configured to rotate between the optical transmitter and the optical receiver to block or conduct transmission of light therebetween.

13. The physiotherapy device of claim 12, wherein the detection unit comprises a plurality of detection portions evenly spaced along a circumferential direction of the sleeve component, an avoidance gap provided between two adjacent detection portions for light to pass through, the plurality of detection portions configured to pass through a light channel between the optical transmitter and the optical receiver in turn.

14. A physiotherapy device, comprising:
a mounting base having a liquid outlet channel with an outlet;
a mounting shaft fixed to the mounting base;
a sleeve component rotatably mounted on the mounting shaft, the sleeve component having an outer peripheral surface with a microneedle assembly configured for contact with skin, the outer peripheral surface positioned proximate to the outlet such that liquid from the liquid outlet channel is applied to the outer peripheral surface;
a switch component rotatably connected to the mounting base, the switch component configured to connect to a container bottle and to switch between a first position in which the switch component connects the liquid outlet channel to the container bottle and a second position in which the switch component disconnects the liquid outlet channel from the container bottle.

15. The physiotherapy device of claim 14, further comprising a limiting cover detachably connected to an end of the mounting shaft away from the mounting base, the limiting cover configured to limit axial movement of the sleeve component along the mounting shaft.

16. The physiotherapy device of claim 14, wherein the switch component is rotatably assembled on a side of the mounting base configured to connect with the container bottle, and the switch component is configured to rotate relative to the mounting base to connect or disconnect the liquid outlet channel and the container bottle.

17. The physiotherapy device of claim 16, wherein the mounting base is provided with a first insertion tube communicating with the liquid outlet channel, the first insertion tube having a first notch on a tube wall thereof; the switch component is provided with a slot having a bottom wall, a second insertion tube and a liquid inlet cut arranged at intervals on the bottom wall; the first insertion tube disposed in the slot, the second insertion tube rotatably disposed in the first insertion tube and having a second notch on a tube wall thereof; in the first position, the second notch positioned opposite to the first notch; and in the second position, the second notch offset from the first notch such that the tube wall of the second insertion tube blocks the first notch.

18. The physiotherapy device of claim 15, wherein the outer peripheral surface of the sleeve component is provided with a smearing portion in contact with a ball, the smearing portion spaced apart from the microneedle assembly, and a plurality of liquid guide grooves alternately arranged with the microneedle assembly, at least part of the liquid guide grooves connected to the smearing portion.

19. The physiotherapy device of claim 18, further comprising a cooling assembly mounted on the end of the mounting shaft away from the mounting base, the cooling assembly comprising a semiconductor cooling component having a cold end and a hot end disposed opposite to each other, the cold end thermally coupled to the limiting cover to cool the limiting cover, the limiting cover having a cold compress surface configured for contact with skin.

20. The physiotherapy device of claim 19, wherein the mounting shaft defines a mounting cavity, and the mounting shaft is formed from a heat-insulating material, an outer peripheral wall of the mounting shaft provided with shaft holes connecting the mounting cavity and the sleeve component, an air inlet gap provided between the limiting cover and the mounting shaft communicating with the mounting cavity, and a fan assembly located in the mounting cavity configured to draw air from the air inlet gap and discharge air through the shaft holes.
